(19) United States
(12) Patent Application Publication
(10) Pub. No.: US 2026/0219194 A1
(43) Pub. Date: Jul. 30, 2026

(54) BIOSENSOR FOR WATER ANALYSIS

(71) Applicant: BIOCELL ANALYTICA UPPSALA AB, Uppsala (SE)

(72) Inventors: Johan LUNDQVIST, Uppsala (SE); Agneta OSKARSSON, Uppsala (SE)

(21) Appl. No.: 19/142,657

(22) PCT Filed: Dec. 21, 2023
(86) PCT No.: PCT/SE2023/051300

§ 371 Date: Jun. 24, 2025

(30) Foreign Application Priority Data
Jan. 4, 2023 (SE) 2350003-6

(51) Int. Cl. G01N 21/76; G01N 21/94; G01N 33/18; G01N 33/50; G01N 33/68

(52) U.S. Cl. CPC G01N 21/763; G01N 21/94; G01N 33/1866; G01N 33/5014; G01N 33/68; G01N 2333/90241

(57) ABSTRACT

An effect-based biosensor for water analysis includes a cell insert including cultures of reporter cells genetically modified to respond to presence of hazardous compounds by expressing a reporter protein and cultures of control cells genetically modified to constitutively express the reporter protein. A first culture of reporter cells and a first culture of control cells are exposed to a water sample to be analyzed, whereas other cultures of reporter cells are exposed to a control water sample and reference water sample(s) and another culture of control cells is exposed to the control water sample. A detector generates values representative of the amount of the reporter protein expressed by the different cultures and a processor generates an output representative of a combined toxic effect of any hazardous compounds in the water sample by processing the values generated by the detector.

### TECHNICAL FIELD

The present invention generally relates to a biosensor, and in particular an effect-based biosensor for water analysis of chemical contaminants.

### BACKGROUND

Humans and the environment are constantly exposed to complex and ever-changing mixtures of both naturally occurring and synthetic chemicals. Some of these chemicals are hazardous and can cause adverse effects on human health and/or the ecosystem. Chemical hazards in water (e.g., contamination by endocrine disruptive compounds) is gaining more and more attention, e.g., in the proposed revision of the European Water Framework Directive and in the recently adopted European drinking water directive. Both drinking water producers and wastewater treatment facilities need early warning systems to detect such chemical hazards in their waters.

The traditional approach in water analysis has been to chemically analyze the respective concentrations of a limited number of selected substances in a water sample. However, these selected substances only account for a very small proportion of the potential toxic effects. A large proportion of the observed unwanted biological effects in water samples is caused by unknown chemicals and/or mixture effects, which can emerge when an organism is exposed to multiple chemicals at the same time.

There is, thus, a need for a water analysis to detect the combined effects of all hazardous chemicals present in a water sample rather than analyzing the concentrations of a few selected substances. It is in particular a need for such a water analysis that can be provided on site at the user and that could be used, for instance, for continuous or at least intermittent monitoring of water quality.

Bioengineered (2012) 3 (2): 124-128 discloses chip-integrated luminescent recombinant reporter bacteria combined with fluidics and light detection systems to form a real-time water biomonitor. Sensors and Actuators B (2016) 225:249-257 discloses a smartphone-based bioluminescence whole-cell toxicity biosensor. Genetically engineered human embryonic kidney cells constitutively expressing a luciferase were used as sentinel cells to investigate cell viability and integrated into 3D printed ready-to-use cartridges, also containing assay reagents. Analytical and Bioanalytical Chemistry (2016) 408:8859-8868 is a further development of the smartphone-based bioluminescence biosensor to not only measure cell viability but also inflammatory activity, whereas the smartphone-based bioluminescence biosensor in Analytical and Bioanalytical Chemistry (2018) 410:1237-1246 is capable of detecting endocrine disruptors. Analytical and Bioanalytical Chemistry (2019) 411:4937-4949 discloses two yeast biosensors stably expressing human estrogen receptors α and β and employing NanoLuc® as a reporter protein to upgrade the widely used yeast estrogen screening (YES) assays. A viability control Saccharomyces cerevisiae strain expresses a chimeric green-emitting luciferase, PLG2. Analytica Chimica Acta (2022) 1200:339583 provides a review of portable light detectors for bioluminescence biosensing applications. Environmental Science & Technology (2022) 56 (20): 14350-14360 discloses a portable nuclear receptor (NR)-based biosensor for analyses of endocrine-disrupting chemicals in environmental water samples.

The above-mentioned portable biosensors enable on-site water sample analysis. However, these biosensors lack control features required to verify that the output of the biosensors is indeed reliable and meaningful.

### SUMMARY

It is a general objective to provide an effect-based biosensor for water analysis of chemical contaminants.

It is a particular objective to provide an effect-based biosensor comprising control features verifying that the output of the biosensors is indeed reliable and meaningful.

These and other objectives are met by the present embodiments.

The present invention is defined in the independent claim. Further embodiments of the invention are defined in the dependent claims.

An aspect of the invention relates to an effect-based biosensor for water analysis. The effect-based biosensor comprises a cell insert comprising multiple cultures of reporter cells genetically modified to respond to presence of hazardous compounds by expressing a reporter protein and multiple cultures of control cells genetically modified to constitutively express the reporter protein. The effect-based biosensor also comprises an inlet system in fluid communication with the cell insert. The inlet system comprises at least one first inlet configured to receive a water sample to be analyzed and in fluid communication with a first culture of the multiple cultures of reporter cells and a first culture of the multiple cultures of control cells. The inlet system also comprises at least one second inlet configured to receive a control water sample and in fluid communication with a second culture of the multiple cultures of reporter cells and a second culture of the multiple cultures of control cells. The inlet system further comprises a third inlet configured to receive a first reference water sample comprising a first concentration of a reference substance and in fluid communication with a third culture of the multiple cultures of reporter cells. The effect-based biosensor further comprises a detector configured to generate, for each culture of the multiple cultures of reporter cells, a value representative of an amount of the reporter protein expressed by the reporter cells and, for each culture of the multiple cultures of control cells, a value representative of an amount of the reporter protein expressed by the control cells. The effect-based biosensor additionally comprises a processor connected to the detector and configured to process the values generated by the detector and generate, based on the value representative of the amount of the reporter protein expressed by the reporter cells in the first culture of the multiple cultures of reporter cells, an output representative of a combined toxic effect of any hazardous compounds in the water sample if the values representative of the amounts of the reporter protein expressed by the control cells in the first culture and in the second culture of the multiple cultures of control cells meet a first condition, and if the values representative of the amounts of the reporter protein expressed by the reporter cells in the second culture and in the third culture of the multiple cultures of reporter cells meet a second condition.

The effect-based biosensor of the invention can be used to analyze the quality of various water samples to investigate whether a combined toxic effect of any hazardous compounds is at acceptable or unacceptable levels. The effect-based biosensor can be used on-site, such as at a drinking water production plant or wastewater treatment plant, and could be designed to be portable. The effect-based biosensor includes internal controls to verify that the output delivered by the biosensor is reliable and meaningful.

### BRIEF DESCRIPTION OF THE DRAWINGS

The embodiments, together with further objects and advantages thereof, may best be understood by making reference to the following description taken together with the accompanying drawings, in which:

FIGS. 1A and 1B schematically illustrate an effect-based biosensor according to an embodiment as seen in a front view (1A) and a side view (1B);

FIG. 2 schematically illustrates an effect-based biosensor according to another embodiment;

FIG. 3 schematically illustrates components of an effect-based biosensor according to various embodiments;

FIGS. 4A to 4C schematically illustrate presentation of output information according to various embodiments;

FIG. 5 illustrates measured luminescence in reporter cells as compared to no cell control and non-transfected cell control;

FIG. 6 illustrates measured luminescence in reporter cells in response to increasing concentrations of 17β-estradiol (E2); and

FIG. 7 illustrates measured luminescence in reporter cells exposed to water samples with low or high estrogen levels.

### DETAILED DESCRIPTION

The present invention generally relates to a biosensor, and in particular an effect-based biosensor for water analysis of chemical contaminants.

The biosensor of the invention is effect-based, which means that the biosensor is capable of determining the combined toxic effect of hazardous compounds that may be present in a water sample. Accordingly, the biosensor uses an effect-based water analysis that integrates the effects of both known and unknown chemicals as well as potential mixture or combined effects of multiple, i.e., at least two, such chemicals. This means that the biosensor can be used for water analysis without prior knowledge of which hazardous compounds, if any, that may be present in the water sample but rather looks at the combined toxic effect that such hazardous compounds may exert.

With reference to FIGS. 1-3, an aspect of the invention relates to an effect-based biosensor 1 for water analysis. The effect-based biosensor 1 comprises a cell insert 10 comprising multiple cultures 11, 13, 15, 16 of reporter cells genetically modified to respond to presence of hazardous compounds by expressing a reporter protein. The cell insert 10 also comprises multiple cultures 12, 14 of control cells genetically modified to constitutively express the reporter protein.

The effect-based biosensor 1 also comprises an inlet system 30 in fluid communication with the cell insert 10. The inlet system 30 comprises at least one first inlet 31 configured to receive a water sample to be analyzed. The at least one first inlet 31 is in fluid communication with a first culture 11 of the multiple cultures 11, 13, 15, 16 of reporter cells and a first culture 12 of the multiple cultures 12, 14 of control cells.

The inlet system 30 also comprises at least one second inlet 33 configured to receive a control water sample. This at least one second inlet 33 is in fluid communication with a second culture 13 of the multiple cultures 11, 13, 15, 16 of reporter cells and a second culture 14 of the multiple cultures 12, 14 of control cells. The inlet system 30 further comprises a third inlet 35 configured to receive a first reference water sample comprising a first concentration of a reference substance. This third inlet 35 is in fluid communication with a third culture 15 of the multiple cultures 11, 13, 15, 16 of reporter cells.

The effect-based biosensor 1 further comprises a detector 50 configured to generate, for each culture 11, 13, 15, 16 of the multiple cultures 11, 13, 15, 16 of reporter cells, a value representative of an amount of the reporter protein expressed by the reporter cells and generate, for each culture 12, 14 of the multiple cultures 12, 14 of control cells, a value representative of an amount of the reporter protein expressed by the control cells.

The effect-based biosensor 1 additionally comprises a processor 70 connected to the detector 50 and configured to process the values generated by the detector 50 and generate, based on the value representative of the amount of the reporter protein expressed by the reporter cells in the first culture 11 of the multiple cultures 11, 13, 15, 16 of reporter cells, an output representative of a combined toxic effect of any hazardous compounds in the water sample if the values representative of the amounts of the reporter protein expressed by the control cells in the first culture 12 and in the second culture 14 of the multiple cultures 12, 14 of control cells meet a first condition, and if the values representative of the amounts of the reporter protein expressed by the reporter cells in the second culture 13 and in the third culture 15 of the multiple cultures 11, 13, 15, 16 of reporter cells meet a second condition.

The cell-insert 1 of the effect-based biosensor 1 comprises multiple cultures 11, 12, 13, 14, 15, 16 of different cells, the reporter cells and the control cells. Both these types of cells express a reporter protein.

“Reporter protein” as used herein indicates any protein that can be constitutively expressed by the control cells and inducibly expressed by the reporter cells, and additionally can be detected, directly or indirectly, by the detector 50. Such a reporter protein is encoded by a so-called reporter gene. Illustrative, but non-limiting, examples of such reporter proteins encoded by reporter genes include β-galactosidase encoded by lacZ, chloramphenicol acetyltransferase encoded by cat, green, red or blue fluorescent protein encoded by gfp, rdp or bfp, luciferase enzyme encoded by luc, etc. The present invention is, however, not limited to such reporter proteins and genes but could be used in connection with any reporter protein or gene that could be expressed by the reporter and control cells and detected by the detector 50. In particular, the reporter protein is a fluorescent protein, a luminescent protein, or produces a detectable fluorescence signal, a detectable luminescence signal or a detectable color.

The reporter cells are genetically modified to respond to presence of hazardous compounds by expressing the reporter gene and thereby produce the reporter protein. These reporter cells thereby have an inducible expression of the reporter gene when exposed to hazardous compounds. The control cells are instead genetically modified to constitutively express the reporter gene and thereby the reporter protein. These control cells are thereby genetically modified to express the reporter gene and produce the reporter protein independent on presence of any hazardous compounds, i.e., independently on whether the control cells are exposed to hazardous compounds or not.

“Hazardous compound” as used herein include any chemical, substance, molecule or indeed composition that may cause adverse effects to humans and/or animals, or the environment in general, when exposed to a water sample comprising such a hazardous compound.

In a particular embodiment, the “hazardous compounds” have a defined mode of action on the reporter cells. This means that such hazardous compounds having a defined mode of action activate a toxicity pathway in the reporter cells when the reporter cells are exposed to such hazardous compounds having the defined mode of action. In such a case, the expression of the reporter protein is induced and take place upon activation of the given toxicity pathway in the reporter cells. The expression of the reporter protein can be triggered and induced at any event in the toxicity pathway but is preferably induced at an initiating molecular event of the toxicity pathway. Such an initiating molecular event is generally an early or upstream event of the toxicity pathway. Such an “initiating molecular event”, also referred to as molecular initiating event (MIE) in the art, is the initial interaction between a molecule (“hazardous compound”) and a biomolecule or biosystem (“reporter cell”) that can be causally linked to an adverse outcome via a pathway (“toxicity pathway”, also referred to as adverse outcome pathway (AOP)).

The inducible expression of the reporter gene in the reporter cells can be achieved by having the reporter gene under transcriptional control of an inducible promoter. Transcription of the reporter gene is thereby induced by the inducible promoter when the reporter cells are exposed to hazardous compounds, and in particular such hazardous compounds having a mode of action resulting in the activation of a defined toxicity pathway or AOP, and wherein activation of the inducible promoter is a molecular event of the toxicity pathway or AOP, preferably an initiating molecular event or MIE. As an example, the inducible promoter may comprise one or multiple response elements. Such response elements are generally short nucleotide sequences within a promoter or an enhancer region that are able to bind specific transcription factors and thereby regulate transcription of the reporter gene controlled by the promoter. Hence, inducible expression could be achieved by using a promoter having one or multiple such response elements and/or by a promoter operatively linked to an enhancer region having one or multiple such response elements. An enhancer region operatively linked to a promoter as used herein indicates that the enhancer is located in vicinity of the promoter, typically up to 1 Mbp away from the promoter, either upstream or downstream, and is capable of controlling transcription of the reporter gene. An enhancer region typically increases the likelihood of transcription of the reporter gene upon binding of a transcription factor to the enhancer region.

Correspondingly, the constitutive expression of the reporter gene in the control cells can be achieved by having the reporter gene under transcriptional control of a constitutive promoter, i.e., an unregulated promoter that allows for continual or constitutive transcription of the reporter gene. The particular promoter used in the control cells for expression of the reporter gene and production of the reporter protein is preferably selected based on the particular type of control cells, such as yeast cells, fish cells or mammalian cells, such as human cells. Illustrative, but non-limiting, examples of such promoters that could be used to control expression of the reporter gene and production of the reporter protein in the control cells include the cytomegalovirus (CMV) promoter, the thymidine kinase (TK) promoter, the simian virus 40 (SV40) promoter, the phosphoglycerate kinase (PGK) promoter, the polyubiquitin C (UBC) promoter, the elongation factor-1 alpha (EF-1α) promoter, and the CAG promoter for mammalian cells, and the PGK promoter, the glyceraldehyde-3-phophate dehydrogenase (TDH3) promoter, the triose phosphate isomerase (TPI1) promoter, the enolase (ENO2) promoter, the alcohol dehydrogenase (ADH1) promoter, and a translational elongation factor EF-1 alpha (TEF1, TEF2) promoter for yeast cells, and the CMV promoter for fish cells.

Expressing the reporter protein as used herein, e.g., an inducible expression of the reporter protein by the reporter cells and a constitutive expression of the reporter protein by the control cells, means that the reporter cells and the control cells are capable of producing the reporter protein, i.e., transcribing the reporter gene to produce a messenger ribonucleic acid (mRNA) and translating the mRNA into the reporter protein.

In an embodiment, the reporter cells comprise a reporter gene encoding the reporter protein. The reporter gene is under transcriptional control of an inducible promoter configured to induce expression of the reporter gene when the reporter cells are exposed to hazardous compounds. In this embodiment, the control cells comprise the reporter gene under transcriptional control of a constitutive promoter.

In an embodiment, the reporter cells are genetically modified to respond to presence of hazardous compounds having a common mode of action resulting in activation of an adverse outcome pathway in the reporter cells.

In a particular embodiment, the reporter cells comprise a reporter gene encoding the reporter protein. The reporter gene is under transcriptional control of an inducible promoter. In this embodiment, activation of the inducible promoter is a molecular event, preferably an initiating molecular event, of the adverse outcome pathway.

The inlet system 30 of the effect-based biosensor 1 is in fluid communication with the cell insert 10. “Fluid communication” or “fluid connection” as used herein indicates that fluid, in particular a liquid, can be transferred between the devices that are in fluid communication or connection with each other. The two devices could be directly connected to each other or could be indirectly connected to each other by a fluid passage allowing transfer of the fluid, preferably liquid, between the devices. As an example of the latter, the previously mentioned inlets 31, 33, 35, 36 of the inlet system 30 could be connected to one or multiple culture chambers 21-26 comprising the cultures 11-16 of reporter cells or control cells though fluid channels 41-46, tubes, pipes, or other passages allowing liquid samples added to the inlets 31, 33, 35, 36 to be transported to the cultures 11-16 in the culture chambers 21-26.

The at least one first inlet 31 is configured to receive a water sample to be analyzed. This at least one first inlet 31 is in fluid communication with a first culture 11 of reporter cells and a first culture 12 of control cells. In an embodiment, the water sample is injected into or otherwise added to a single inlet 31 and is then routed by respective fluid channels 41, 42 to the first cultures 11, 12 of reporter cells and control cells. Alternatively, the cell insert 10 could comprise two such first inlets 31, one in fluid communication with the first culture 11 of reporter cells and the other in fluid communication with the first culture 12 of control cells. In such an embodiment, the water sample is added to both these first inlets 31.

Correspondingly, the at least one second inlet 33 is configured to receive a control water sample. This control water sample is preferably a pure water sample, i.e., a water sample lacking any hazardous compounds. The control water sample is thereby preferably a purified water sample, such as a Milli-Q® water sample or another reference water standard. The purified water sample could be produced by any water purification process capable of removing hazardous compounds from water including, but not limited to, distillation, capacitive deionization, reverse osmosis, carbon filtering, microfiltration, ultrafiltration, and/or electrodeionization.

The at least one second inlet 33 is in fluid communication with a second culture 13 of reporter cells and a second culture 14 of control cells. In an embodiment, the control water sample is injected into or otherwise added to a single inlet 33 and is then routed by respective fluid channels 43, 44 to the second cultures 13, 14 of reporter cells and control cells. Alternatively, the cell insert 10 could comprise two such second inlets 33, one in fluid communication with the second culture 13 of reporter cells and the other in fluid communication with the second culture 14 of control cells. In such an embodiment, the control water sample is added to both these second inlets 33.

The third inlet 35 of the cell insert 10 is configured to receive a first reference water sample comprising a first concentration of a reference substance. The third inlet 35 is in fluid communication, such as with a fluid channel 45, with a third culture 15 of reporter cells. The reference substance present at the first concentration in this first reference water sample is then capable of inducing expression of the reporter protein by the third culture 15 of reporter cells. Thus, the reporter cells in this third culture 15 respond to the reference substance by expressing the reporter gene and thereby by producing the reporter protein. In other words, the reference substance is capable of, directly or indirectly, activating the inducible promoter that controls transcription of the reporter gene in the third culture 15 of reporter cells.

In an embodiment, the reference substance is capable of activating the previously mentioned adverse outcome pathway in the reporter cells. In other words, the reference substance preferably has the same common mode of action as any hazardous compounds that may be present in the water sample.

The detector 50 of the effect-based biosensor 1 is capable of detecting the presence of the reporter protein as produced by the cultures 11, 13, 15, 16 of reporter cells and the cultures 12, 14 of control cells. The detector 50 then generates values representative of the respective amount of reporter protein expressed by the reporter cells and the control cells. Hence, the detector 50 is configured to generate a value V_{R1} representative of the amount of reporter protein expressed by the first culture 11 of reporter cells, a value V_{R2} representative of the amount of reporter protein expressed by the second culture 13 of reporter cells, a value V_{R3} representative of the amount of reporter protein expressed by the third culture 15 of reporter cells, a value V_{C1} representative of the amount of reporter protein expressed by the first culture 12 of control cells, and a value V_{C2} representative of the amount of reporter protein expressed by the second culture 14 of control cells.

The processor 70 is connected to the detector 50 and is configured to process these values generated by the detector 50. For instance, the processor 70 and the detector 50 could be connected through a communication or data bus 76, over which data can be forwarded between devices, units or modules of the effect-based biosensor 1. Alternatively, the detector 50 is connected, directly or indirectly, such as over the data bus 76, to a memory 72. In such a case, the values generated by the detector 50 could be stored in the memory 72. In such a case, the processor 70 could retrieve the values from the memory 72.

The optional memory 72 of the effect-based biosensor 1 may comprise a computer program comprising instructions, which when executed by the processor 70, cause the processor 70 to process these values generated by the detector 50 and generate, based on the value representative of the amount of the reporter protein expressed by the reporter cells in the first culture 11 of reporter cells, an output representative of a combined toxic effect of any hazardous compounds in the water sample if the values representative of the amounts of the reporter protein expressed by the control cells in the first culture 12 and in the second culture 14 of control cells meet a first condition, and if the values representative of the amounts of the reporter protein expressed by the reporter cells in the second culture 13 and in the third culture 15 of reporter cells meet a second condition.

Thus, the processing of the output from the detector 50 could be implemented in a computer program, which is loaded into the memory 72 for execution by the processor 70. In such an embodiment, the processor 70 and the memory 72 are interconnected to each other to enable normal software execution, such as over the communication or data bus 76.

The term processor 70 should be interpreted in a general sense as any circuitry, system or device capable of executing program code or computer program instructions to perform a particular processing, determining or computing task. The processor 70 is, thus, configured to perform, when executing the computer program, well-defined processing tasks such as those described herein.

In an embodiment, the processor 70 is configured to generate an output representative of a combined toxic effect of any hazardous compounds in the water sample based on the value V_{R1} representative of the amount of the reporter protein expressed by the reporter cells in the first culture 11 of reporter cells if two conditions are met. Thus, the effect-based biosensor 1 includes an internal evaluation of the values as generated by the detector 50 to verify that the effect-based biosensor 1 outputs a meaningful and relevant result of the water analysis. This means that the effect-based biosensor 1 is capable of verifying that the output is indeed reliable and preferably only generates the output if, and preferably only if, the two conditions are met.

Hence, the processor 70 verifies whether the values V_{C1}, V_{C2} representative of the amounts of the reporter protein expressed by the control cells in the first and second cultures 12, 14 of control cells meet a first condition and whether the values V_{R2}, V_{R3} representative of the amounts of the reporter protein expressed by the reporter cells in the second and third cultures 13, 15 of reporter cells meet a second condition.

The first condition is a so-called cell viability condition. The control cells in the two cultures 12, 14 of control cells constitutively express the reporter protein. The difference between these two cultures 12, 14 of control cells is that the first culture 12 of control cells has been exposed to the water sample to be analyzed, whereas the second culture 14 of control cells has been exposed to the control water sample. Any significant difference between the values V_{C1}, V_{C2} is thereby mainly due to a difference in cell viability between the two cultures 12, 14 of control cells. In particular, this control test verifies that the water sample as such is not cytotoxic and thereby reduces the viability of the control cells. In such a situation, the water sample would also be cytotoxic to the reporter cells and cause a reduction in the viability of the reporter cells in the first culture 11 exposed to the water sample. Such cytotoxic effects to the reporter cells by the hazardous compounds may mask specific molecular events caused by the hazardous compounds on the reporter cells. Such a reduction of the viability of the reporter cells in the first culture 11 of reporter cells would then lead to a “lower than expected” expression of the reporter protein in the first culture 11 of reporter cells since the first culture 11 contains “less than expected” viable reporter cells that are capable of producing the reporter protein. Thus, the value V_{R1} as generated by the detector 50 would not be representative of the true combined toxic effect of any hazardous compounds in the water sample but would instead be misleading and flawed by the low cell viability of the reporter cells in the first culture 11 of reporter cells. In other words, the first condition is employed to verify that the reporter cells exposed to the water sample to be analyzed are sufficiently viable to get a meaningful interpretation of the value V_{R1} generated by the detector 50 for the reporter cells in the first culture 11 of reporter cells.

Accordingly, the first condition or the cell viability condition is preferably met if the difference between the values V_{C1}, V_{C2} is within an acceptable range, such as |V_{C2}−V_{C1}|≤T₁ or

$${T}_{2} \leq \frac{{V}_{C 1}}{{V}_{C 2}}$$

for some predefined threshold values T₁, T₂. In an embodiment, T₂≤1.

Hence, in an embodiment, the processor 70 of the effect-based biosensor 1 is configured to compare the values V_{C1}, V_{C2} representative of the amounts of the reporter protein expressed by the control cells in the first culture 12 and the second culture 14 of the multiple cultures 12, 14 of control cells. The processor 70 is, in this embodiment, also configured to determine that the first condition is met if the value V_{C1} representative of the amount of the reporter protein expressed by the control cells in the first culture 12 of the multiple cultures 12, 14 of control cells is at least a predefined percentage of the value V_{C2} representative of the amount of the reporter protein expressed by the control cells in the second culture 14 of the multiple cultures 12, 14 of control cells.

This predefined percentage is typically dependent on the particular control cells used, the type of water sample analysis performed by the effect-based biosensor 1, i.e., the particular mode of action of the hazardous compounds. In an illustrative, but non-limiting, embodiment, the predefined percentage is at least 70%, preferably at least 75%, and more preferably at least 80%. These illustrative embodiments correspond to setting the threshold T₂ to 0.70, 0.75 or 0.80.

In an embodiment, the first condition is a cell viability condition. In a particular embodiment, the cell viability condition indicates that the water sample is not cytotoxic for the reporter cells and the control cells.

The second condition involves a comparison of “minimum” and “maximum” values generated by the detector 50. This second condition could thereby be regarded as responsiveness condition. In particular, the value V_{R2} representative of the amount of the reporter protein expressed by the reporter cells in the second culture 13 of reporter cells is representative of the “minimum value” for the effect-based biosensor 1 since this value V_{R2} is generated for reporter cells exposed to the control water sample, such as pure water. Hence, the reporter cells in this second culture 13 are not exposed to any hazardous compounds or other substances that should induce expression of the reporter protein by these reporter cells. Hence, the value V_{R2} represents a background level or minimum value for the effect-based biosensor 1.

Correspondingly, the value V_{R3} representative of the amount of the reporter protein expressed by the reporter cells in the third culture 15 of reporter cells is representative of the “maximum value” for the effect-based biosensor 1. This value V_{R3} is generated for reporter cells exposed to the first concentration of the reference substance. The reference substance is preferably a substance that induces expression of the reporter protein by the reporter cells. The reference substance preferably has the same mode of action as any hazardous compounds that may be present in the water sample to be analyzed. The first concentration of this reference substance is then preferably selected to achieve a higher expression of the reporter protein by the reporter cells in the third culture 15 than what is expected to be produced by the reporter cells in the first culture 11 and that are exposed to the water sample to be analyzed. Hence, the value V_{R3} represents a maximum value for the effect-based biosensor 1.

The second condition is preferably met if the value V_{R3} (“maximum value”) is larger than the value V_{R2} (“minimum value”), and in particular is at least a predefined number of times larger than V_{R2}, such as VR3>k×V_{R2}, wherein k is a positive number larger than 1.

In an embodiment, the processor 70 of the effect-based biosensor 1 is configured to compare the values representative of the amounts of the reporter protein expressed by the reporter cells in the second culture 13 and in the third culture 15 of the multiple cultures 11, 13, 15, 16 of reporter cells. The processor 70 is also, in this embodiment, configured to determine that the second condition is met if the value representative of the amount of the reporter protein expressed by the reporter cells in the third culture 15 of the multiple cultures 11, 13, 15, 16 of reporter cells is at least a predefined number of times larger than the value representative of the amount of the reporter protein expressed by the reporter cells in the second culture 13 of the multiple cultures 11, 13, 15, 16 of reporter cells.

The value V_{R2} could thereby be regarded as minimum control value, whereas the value V_{R3} could then be regarded as the maximum control value.

The actual value of the number k is at least partly dependent on the particular reporter cells, the reporter protein and the detector 50.

In a particular embodiment, the predefined times (number k) is at least 2, preferably at least 3 and more preferably at least 4. For instance, the predefined times (number k) could be selected within an interval of from 2 up to 30, preferably selected within an interval of from 3 up to 30, and more preferably selected within an interval of from 4 up to 30. In a particular embodiment, the predefined times (number k) could be selected within an interval of from 2 up to 10, preferably selected within an interval of from 3 up to 10, and more preferably selected within an interval of from 4 up to 10.

The second condition thereby verifies that the effect-based biosensor 1 is responsive and can produce different values representing different amounts of expressed reporter protein. If the difference between these maximum and minimum control values is less than expected (V_{R3}<k×V_{R2}) then a meaningful interpretation of the value generated by the detector 50 for the reporter cells in the first culture 11 of reporter cells is not possible.

In an embodiment, the second condition is a responsiveness condition. In a particular embodiment, the responsiveness condition indicates that the effect-based biosensor 1 can produce range of values representative of different amounts of reporter protein.

In an embodiment, the processor 70 is configured to generate, based on the value representative of the amount of the reporter protein expressed by the reporter cells in the first culture 11 of the multiple cultures 11, 13, 15, 16 of reporter cells, the output if the values representative of the amounts of the reporter protein expressed by the control cells in the first culture 12 and in the second culture 14 of the multiple cultures 12, 14 of control cells meet the first condition, if the values representative of the amounts of the reporter protein expressed by the reporter cells in the second culture 13 and in the third culture 15 of the multiple cultures 11, 13, 15, 16 of reporter cells meet the second condition, and if the values representative of the amount of the reporter protein expressed by the reporter cells in the first culture 11 and the second culture 13 of the multiple cultures 11, 13, 15, 16 of reporter cells meet a third condition.

Hence, in this embodiment, the processor 70 of the effect-based biosensor 1 further verifies whether a third condition is met. This third condition involves determining whether the value V_{R1} representative of the amount of reporter protein expressed by the reporter cells in the first culture 11 of reporter cells is equal to or above the value V_{R2} representative of the amount of reporter protein expressed by the reporter cells in the second culture 13 of reporter cells. In other words, this third condition investigates whether the reporter cells exposed to the water sample to be analyzed express an equal amount or more reporter protein than the reporter cells exposed to the control water sample, such as pure water. Hence, this third condition is met if V_{R1}>V_{R2}. This third condition therefore verifies that the value determined by the detector 50 for the reporter cells in the first culture 11 of reporter cells is at least equal to the minimum control value.

In an embodiment, the processor 70 of the effect-based biosensor 1 is configured to compare the value representative of the amount of the reporter protein expressed by the reporter cells in the first culture 11 of the multiple cultures 11, 13, 15, 16 of reporter cells with the value representative of the amount of the reporter protein expressed by the reporter cells in the second culture 13 of the multiple cultures 11, 13, 15, 16 of reporter cells. The processor 70 is in this embodiment also configured to determine that the third condition is met if the value representative of the amount of the reporter protein expressed by the reporter cells in the first culture 11 of the multiple cultures 11, 13, 15, 16 of reporter cells is equal to or above the value representative of the amount of the reporter protein expressed by the reporter cells in the second culture 13 of the multiple cultures 11, 13, 15, 16 of reporter cells.

In a particular embodiment, this third condition involves determining whether the value V_{R1} is within the defined range of the effect-based biosensor 1, i.e., within the range as defined by the minimum control value (V_{R2}) and the maximum control value (V_{R2}). Hence, in this particular embodiment, the third condition is met if V_{R2}≤V_{R1}≤V_{R3}, i.e., V_{R1∈}[V_{R2}, V_{R3}].

In this particular embodiment, the processor 70 is configured to determine that the third condition is met if the value representative of the amount of the reporter protein expressed by the reporter cells in the first culture 11 of the multiple cultures 11, 13, 15, 16 of reporter cells is equal to or below the value representative of the amount of the reporter protein expressed by the reporter cells in the third culture 15 of the multiple cultures 11, 13, 15, 16 of reporter cells but equal to or above the value representative of the amount of the reporter protein expressed by the reporter cells in the second culture 13 of the multiple cultures 11, 13, 15, 16 of reporter cells.

The above particular embodiment is optional since in some situations and depending on the water sample to be analyzed, the particular concentration of the reference compound in the first reference water sample and the mode of action of hazardous compounds as detected by the reporter cells, the value V_{R1} may actually be higher than the value V_{R3}. The effect-based biosensor 1 may still operate correctly and give a relevant output for the water sample to be analyzed.

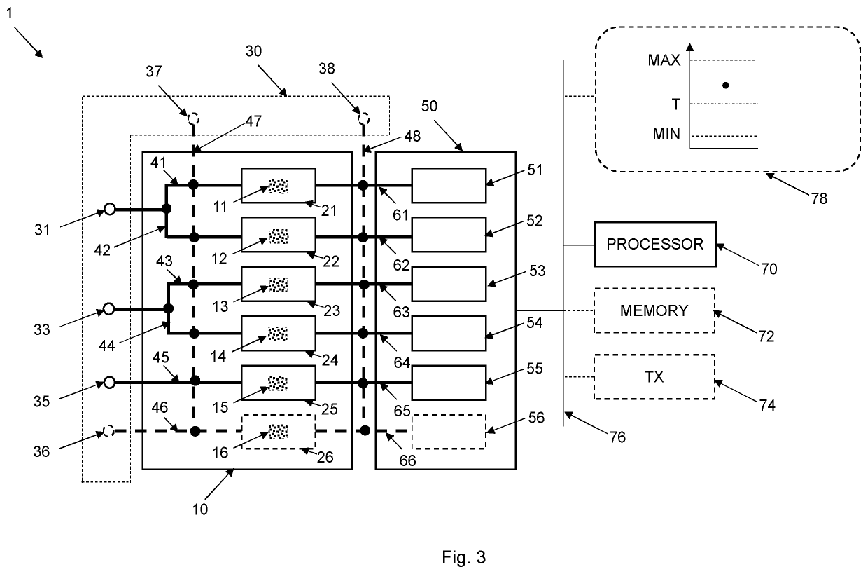

In an embodiment, the inlet system 30 comprises a fourth inlet 36 as indicated in FIG. 3. This fourth inlet 36 is optional but is preferably included to receive a second reference water sample comprising a second concentration of the reference substance that is lower than the first concentration. The fourth inlet 36 is in fluid communication with a fourth culture 16 of the multiple cultures 11, 13, 15, 16 of reporter cells. In such an embodiment, the processor 70 is configured to compare the value representative of the amount of the reporter protein expressed by the reporter cells in the first culture 11 of the multiple cultures 11, 13, 15, 16 of reporter cells and the value representative of the amount of the reporter protein expressed by the reporter cells in the fourth culture 16 of the multiple cultures 11, 13, 15, 16 of reporter cells. The processor 70 is in this embodiment also configured to generate the output based on the comparison if the values representative of the amounts of the reporter protein expressed by the control cells in the first culture 12 and in the second culture 14 of the multiple cultures 12, 14 of control cells meet the first condition, and if the values representative of the amounts of the reporter protein expressed by the reporter cells in the second culture 13 and in the third culture 15 of the multiple cultures 11, 13, 15, 16 of reporter cells meet the second condition.

Thus, in this embodiment, the value V_{R1} representative of the amount of reporter protein expressed by the reporter cells exposed to the water sample to be analyzed is compared to the value V_{R4} representative of the amount of reporter protein expressed by the reporter cells exposed to the second reference water sample comprising the second concentration of the reference substance. The output generated by the processor 70 is then generated based on this comparison.

The reporter cells in this fourth culture 16 of reporter cells are, thus, exposed to the same reference substance as the reporter cells in the third culture 15 of reporter cells but at a lower concentration. This means that whereas the value V_{R3} represents a maximum control value, the value V_{R4} could be regarded as a trigger level or value or acceptable level or value. Thus, if the value V_{R1} is equal to or higher, or alternatively higher, than the value V_{R4} then the combined toxic effect of any hazardous compounds in the water sample is regarded as being higher than the “toxic effect” caused by the second concentration of the reference substance and thereby higher than the trigger or acceptable level. Correspondingly, if the value V_{R1} is lower, alternatively equal to or lower, than the value V_{R4} then the combined toxic effect of any hazardous compounds in the water sample is regarded as less than the “toxic effect” caused by the second concentration of the reference substance and thereby less than the trigger or acceptable level.

In a particular embodiment, the processor 70 is configured to generate an output indicating that the combined toxic effect of any hazardous compounds in the water sample is unacceptably high if the value representative of the amount of the reporter protein expressed by the reporter cells in the first culture 11 of the multiple cultures 11, 13, 15, 16 of reporter cells is higher than, or equal to or higher than, the value representative of the amount of the reporter protein expressed by the reporter cells in the fourth culture 16 of the multiple cultures 11, 13, 15, 16 of reporter cells.

In a particular embodiment, the processor 70 is configured to generate an output indicating that the combined toxic effect of any hazardous compounds in the water sample is acceptable if the value representative of the amount of the reporter protein expressed by the reporter cells in the first culture 11 of the multiple cultures 11, 13, 15, 16 of reporter cells is equal to or lower than, or lower than, the value representative of the amount of the reporter protein expressed by the reporter cells in the fourth culture 16 of the multiple cultures 11, 13, 15, 16 of reporter cells.

Thus, in an embodiment, the water sample to be analyzed is regarded as being acceptable if V_{R1} is less than V_{R4} and otherwise regarded as being unacceptable. In another embodiment, the water sample to be analyzed is regarded as being acceptable if V_{R1} is equal to or less than V_{R4} and otherwise regarded as being unacceptable. The trigger level could be regarded as a level at which actions need to be triggered or taken to purify the sample from hazardous compounds.

The second concentration of the reference substance in the second water sample is preferably determined based on the type of water sample to be analyzed. For instance, the second concentration may be lower for analyzing drinking water than analyzing other types of water samples, such as wastewater. Furthermore, different regions or countries may have different regulations or guidelines regarding the presence of various hazardous compounds in water samples. In such a case, the concentration of the reference substance in the second water sample can be selected based on such regulations or guidelines.

The comparison between the values V_{R1} and V_{R4} in these embodiments could be regarded as investigating whether any hazardous compounds in the water samples produce a (toxic) effect on the reporter cells that is larger than or less than the corresponding effect on the reporter cells as produced by a predefined trigger bioequivalent concentration of the reference substance.

In an embodiment, the processor 70 investigates whether a fourth condition is met. This fourth condition implies that the trigger or acceptance value (V_{R4}) should be within the range defined by the minimum control value (V_{R2}) and the maximum control value (V_{R3}), i.e., whether V_{R2}<V_{R4}<V_{R3}.

In such an embodiment, the processor 70 is configured to generate the output if the values representative of the amounts of the reporter protein expressed by the control cells in the first culture 12 and in the second culture 14 of the multiple cultures 12, 14 of control cells meet the first condition, if the values representative of the amounts of the reporter protein expressed by the reporter cells in the second culture 13 and in the third culture 15 of the multiple cultures 11, 13, 15, 16 of reporter cells meet the second condition, and if the values representative of the amounts of the reporter protein expressed by the reporter cells in the second culture 12, the third culture 15, and the fourth culture 16 of the multiple cultures 11, 13, 15, 16 of reporter cells meet a fourth condition.

In a particular embodiment, the processor 70 is configured to compare the value representative of the amount of the reporter protein expressed by the reporter cells in the fourth culture 16 of the multiple cultures 11, 13, 15, 16 of reporter cells with the values representative of the amounts of the reporter protein expressed by the reporter cells in the second culture 13 and in the third culture 15 of the multiple cultures 11, 13, 15, 16 of reporter cells. The processor 70 is also configured in this embodiment to determine that the fourth condition is met if the value representative of the amount of the reporter protein expressed by the reporter cells in the fourth culture 16 of the multiple cultures 11, 13, 15, 16 of reporter cells is below the value representative of the amount of the reporter protein expressed by the reporter cells in the third culture 15 of the multiple cultures 11, 13, 15, 16 of reporter cells but above the value representative of the amount of the reporter protein expressed by the reporter cells in the second culture 13 of the multiple cultures 11, 13, 15, 16 of reporter cells.

The various embodiments described in the foregoing with regard to the first to fourth conditions and the comparison of the value V_{R1} to the trigger or acceptance value can be combined.

In an embodiment, the cell insert 10 comprises multiple cultures 11, 13, 15, 16 of reporter cells genetically modified to respond to presence of hazardous compounds by expressing and extracellularly releasing the reporter protein and multiple cultures 12, 14 of control cells genetically modified to constitutively express and extracellularly release the reporter protein.

Hence, in this embodiment, the reporter cells and the control cells can express and extracellularly release the reporter protein. This means that the produced reporter protein is transported out of the reporter and control cells and will thereby be present in the medium, in which the reporter and control cells are present.

Extracellularly release or secretion of the reporter cells may, for instance, be achieved by the usage of secretory signal peptides (SPs), which are sequence motifs targeting proteins for translocation across the endoplasmic reticulum membrane and then secreted extracellularly. There are various such SPs that could be used according to the embodiments. The SPs are most commonly present at the N-terminus of proteins targeted for the secretory pathway. Hence, in a particular embodiment, the reporter cells and the control cells encode the reporter protein comprising a SP, preferably at the N-terminus of the reporter protein. The SP may, once translocated across the endoplasmic reticulum membrane, be removed from the reporter protein by a signal peptidase.

In these embodiments, the detector 50 could detect the presence of the reporter proteins extracellularly, i.e., in the media in which the reporter or control cells are present in the different cultures 11-16 in the cell insert 10.

The embodiments are, however, not limited to the usage of reporter and control cells that are capable of not only expressing the reporter protein by also extracellularly secreting or releasing the reporter protein. In another embodiment, the reporter cells and the control cells could instead be lysed following a period of time from adding the various water samples to the inlets 31, 33, 35, 36 of the inlet system 30. For instance, the inlet system 30 could comprise an inlet 37 in fluid communication with all the cultures 11-16 of reporter cells and control cells in the cell insert 10. In such a case, a lysing agent, such as a lysis buffer, could be added to the inlet 37 to lyse the reporter cells and the control cells and release the reporter proteins into the media. A lysis buffer is a buffer solution used for breaking open cells. Most lysis buffers contain buffering salts, e.g., Tris-HCl, and ionic salts, e.g., NaCl, to regulate the pH and osmolarity of the lysate. Optionally, but preferably, the lysis buffer may comprise detergents, such as Triton X-100 or sodium dodecyl sulfate (SDS), to break up membrane structures and/or protease inhibitors to prevent breakdown of the released reporter protein.

In an embodiment, the inlet system 30 further comprises a substrate inlet 38 configured to receive a substrate that is converted by the reporter protein into a product upon emission of light. In such an embodiment, the detector 50 is an optical detector 50 configured to detect emitted light.

The substrate inlet 38 is preferably in fluid communication with the different cultures 11-16 and/or with downstream detector chambers 51-56, which are further described herein. In such a case, the substrate as received in this substrate inlet 38 can be converted by the reporter protein into a product upon emission of light. In such an embodiment, the reporter protein is an enzyme that catalyzes the substrate into a product upon emission of light.

In these embodiments, the reporter protein is preferably a luciferase that produces bioluminescence that can be detected by the optical detector 50.

For instance, furimazine (8-benzyl-2-(furan-2-ylmethyl)-6-phenylimidazo[1,2-a] pyrazin-3-ol) could be used as substrate for the luciferase NanoLuc®. Another example of substrate is X-gal (5-bromo-4-chloro-3-indolyl-β-D-galactopyranoside) that could be used together with β-galactosidase as reporter protein. β-galactosidase will then cleave the glycosidic bond in X-gal and form galactose and 5-bromo-4-chloro-3-hydroxyindole, which dimerizes and oxidizes to 5,5′-dibromo-4,4′-dichloro-indigo that is an intense blue product that can be detected by the detector 50.

The cell insert 30 could comprise a single substrate inlet 38 that is in fluid communication with the multiple cultures 11-16 and/or the downstream detector chambers 51-56 by a fluid channel 48. Alternatively, the cell insert 30 could comprise multiple substrate inlets 38, such as one substrate inlet 38 per culture 11-16 of reporter and control cells. In such a case, each substrate inlet 38 is in fluid communication with a respective culture 11-16 and/or a respective downstream detector chamber 51-56.

In an embodiment, the reporter cells and the control cells are selected from the group consisting of yeast cells, fish cells and mammalian cells. In a preferred embodiment, the reporter cells and the control cells are mammalian cells and more preferably human cells. The control cells are preferably the same type of cells as the reporter cells but with the difference that the control cells are genetically modified to constitutively express the reporter cells but the reporter cells are genetically modified to respond to presence of hazardous compounds by expressing the reporter protein.

Typical human cells that could be used for the reporter and control cells are human cell lines or human cancer cells.

In an embodiment, the inlet system 30 comprises at least one medium inlet 37 configured to receive a culture medium for the reporter cells and the control cells. The at least one medium inlet 37 is in fluid communication with the multiple cultures 11, 13, 15, 16 of reporter cells and the multiple cultures 12, 14 of control cells.

The inlet system 30 could comprise a single medium inlet 37 that is in fluid communication with the multiple cultures 11-16 of reporter and control cells, such as by a fluid channel 47. Alternatively, the inlet system 30 could comprise multiple medium inlets 37, such as one medium inlet per culture 11-16 of reporter and control cells.

The culture medium added to the at least one medium inlet 37 is optionally, but preferably, a concentrated culture medium. The reason for using a concentrated culture medium is that various water samples will be added to the inlets 31, 33, 35, 36 in fluid communication with the cultures 11-16 of the reporter and control cells. These water samples will then dilute the concentrated culture medium to obtain a suitable culture medium for the cultures 11-16 of the reporter and control cells once the concentrated culture medium has been diluted by the different water samples.

In an embodiment, the cell insert 10 comprises a plurality of spatially separated culture chambers 21-26 each comprising a culture 11, 13, 15, 16 of reporter cells or a culture 12, 14 of control cells. The culture chambers 21-26 could be any chambers or structure that can comprise the reporter or control cells and allow the reporter or control cells to express the reporter protein. Illustrative, but non-limiting, examples of such culture chambers 21-26 include culture wells, culture matrices, culture reservoirs, culture channels, etc.

In such an embodiment, the inlets 31, 33, 35, 36 and the optional at least one medium inlet 37 of the cell insert 10 are preferably in fluid communication with the culture chambers 21-26 by various fluid channels 41-47 as discussed in the foregoing.

The optional at least one medium inlet 37 could be used to add the previously mentioned lysing agent to the cultures 11-16 of reporter cells and control cells.

The cell insert 10 is preferably designed to prevent or at least restrict flows and thereby cross-talk between the different cultures 11-16 and thereby between the different culture chambers 21-26. This reduces the risk of flow of reporter protein produced by one culture 11-16 of reporter or control cells in one culture chamber 21-26 from reaching another culture 11-16 of reporter or control cells in another culture chamber 21-26 in the cell insert 10. This could be achieved, for instance, by having separate inlets 31, 33, 35, 36 for the different cultures 11-16 and culture chambers 21-26, possibly including different medium inlets 37. Alternatively, the cell insert 10 could be designed to prevent or at least restrict cross-flow between different cultures 11-16 and culture chambers 21-26, such as by the design of the fluid channels 41-47 and/or by using one-way valves (check valves) in the fluid channels 41-47 to merely allow fluid to flow through the one-way valves in one direction.

The effect-based biosensor 1 may optionally comprise multiple detector chambers 51-56, also referred to as detector cells herein, such as one detector chamber 51-56 per culture 11-16 of reporter or control cells and per optional culture chamber 21-26. In such a case, each detector chamber 51-56 is preferably in fluid communication with a respective culture 11-16 and optional culture chamber 21-26 by a fluid channel 61-66 interconnecting one culture chamber 21-26 with one detector chamber 51-56. The detector 50 is then arranged to detect the reporter protein, a product generated by the reporter protein from an added substrate, or a light or color change produced by the reporter protein from the different detector chambers 51-56. In these cases, the reporter protein or at least the product produced by the reporter protein is moved along the fluid channels 61-66 from the culture chambers 21-26 to the detection chambers 51-56. This movement or transport of the reporter protein and/or product could be achieved by adding fresh culture medium or pure water, such as to the at least one medium inlet 37 to push the produced reporter protein and/or product from the culture chambers 21-26 to the detection chambers 51-56.

In another embodiment, the detection chambers 51-56 are not connected to the culture chambers 21-26 by fluid channels 61-66 but rather by optical fibers or similar structures capable of forwarding light produced by the reporter proteins in the culture chambers 21-26 to the detection chambers 51-56 where the light is detected by the detector 50.

The cell insert 10 could be a separate, preferably disposable part of the effect-based biosensor 1. In such a case, once a water sample has been analyzed by the effect-based biosensor 1, the cell insert 10 is removed from the effect-based biosensor 1 and discarded. A new cell insert 10 is then inserted in the effect-based biosensor 1 to be used for the analysis of a new water sample by the effect-based biosensor 1.

It is also possible to use a cell insert 10 for analysis of multiple water samples if the detector 50 of the effect-based biosensor 1 can detect reporter protein as produced by the multiple cultures 11, 13, 15, 16 of reporter cells and the multiple cultures 12, 14 of control cells without the need for lysing the reporter and control cells. In such an embodiment, fresh culture medium could be added to the at least one medium inlet 37 to, between successive water analysis occasions, flush away and remove any reporter proteins produced by the reporter and control cells during the previous water analysis occasion. In such an embodiment, the effect-based biosensor 1 may comprise at least one outlet in fluid communication with the detection chambers 51-56 and/or the culture chambers 21-26.

The cell insert 10 could be pre-loaded with the multiple cultures 11, 13, 15, 16 of reporter cells and the multiple cultures 12, 14 of control cells. For instance, the reporter cells and the control cells could be present in the previously mentioned culture chambers 21-26 together with a culture medium capable of supporting the reporter and control cells. This culture medium could be the same culture medium as is added to the at least one medium inlet 37 or another culture medium. Alternatively, the reporter cells and control cells could be provided in the cell insert 10 as dried, such as freeze dried, cells, which are then activated and revived upon addition of the culture medium into the at least one medium inlet 37. Such an embodiment involves the use of reporter and control cells that are capable of being freeze dried and then revived.

In an embodiment, the effect-based biosensor 1 preferably comprises an insert receptacle 4, see FIG. 1B, configured to receive and support the cell insert 10. In such a case, the cell insert 10 is releasably supported by the insert receptacle 4. For instance, the effect-based biosensor 1 comprises a housing 2 comprising the different inlets 31, 33, 35, 36, 37, 38 as indicated in FIG. 1A. The housing 2 then comprises the insert receptacle 4 configured to receive and support the cell insert 10. The cell insert 10 could, once it has been used, be removed from the insert receptacle 4 to be replaced by a new, fresh cell insert 10.

In an embodiment, the effect-based biosensor 1 also comprises a display screen 78 configured to display the output generated by the processor 70 or a parameter derived based on the output as shown in FIG. 1A.

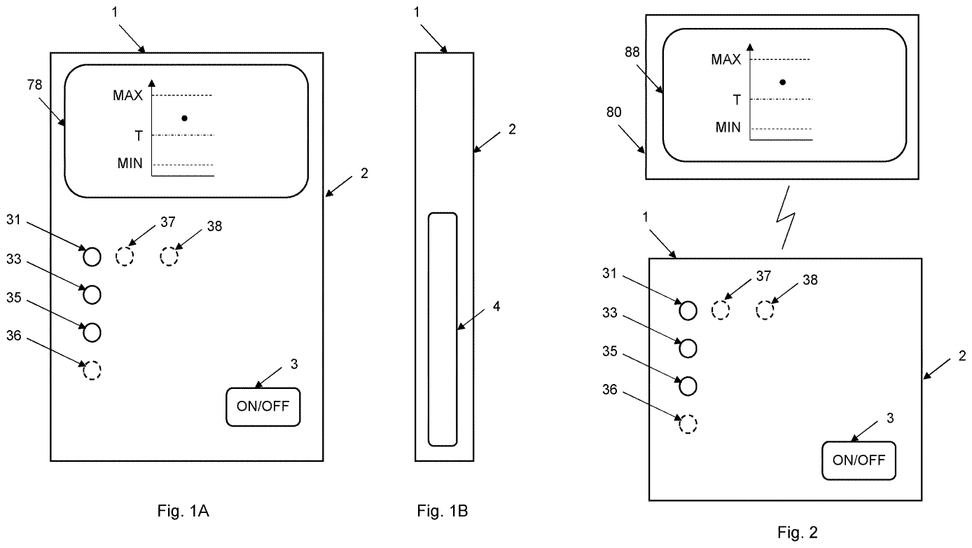

In another embodiment, the effect-based biosensor 1 does not necessarily comprise any display screen, see FIG. 2. In clear contrast, the effect-based biosensor 1 comprises a transmitter 74, see FIG. 3, configured to wirelessly transmit the output or the parameter derived based on the output to an external device 80. This external device 80 then preferably comprises a display screen 88 configured to display the received output or the parameter derived based on the output. The effect-based biosensor 1 could, alternatively, be connected to the external device 80 by a wire.

The effect-based biosensor 1 may comprise a button 3 to turn on the biosensor 1 to initiate an analysis of a water sample and turn off the biosensor 1 once the analysis is completed.

The detector 50 of the effect-based biosensor 1 could be in the form of various types of detectors depending on the particular reporter protein expressed by the reporter and control cells. For instance, an optical detector 50 could be used to detect light or a color change as produced by the reporter protein. Illustrative, but non-limiting, examples of optical detectors 50 include a camera, a charge-coupled device (CCD), various optical or light detectors, such as photoconductive devices, photvoltaic cells and photodiodes, etc. The embodiments are, however, not limited to such optical detectors 50 but also encompass other types of detectors 50, such as protein sensors and biosensors.

As mentioned in the foregoing, the effect-based biosensor 1 is preferably employed to detect presence of any hazardous compounds having a common mode of action in a water sample. Various such common mode of actions are possible including, but not limited, to modification of sex hormone receptors, such as activation or inhibition of such sex hormone receptors, activation or inhibition of other hormone receptors, including, but not limited to, thyroid hormone receptors, glucocorticoid receptors, mineralocorticoid receptors, induction of oxidative stress, activation of aryl hydrocarbon receptor, and genotoxicity.

Hence, in an embodiment, the cell insert 10 comprises multiple cultures 11, 13, 15, 16 of reporter cells genetically modified to respond to presence of hazardous compounds having a common mode of action on the reporter cells by expressing the reporter protein. In this embodiment, the common mode of action is selected from the group consisting of activation of sex hormone receptors, inhibition of sex hormone receptors, activation or inhibition of other hormone receptors, including, but not limited to, thyroid hormone receptors, glucocorticoid receptors, mineralocorticoid receptors, induction of oxidative stress, activation of aryl hydrocarbon receptor, genotoxicity, and any combination thereof.

Compounds present in water samples can activate or inhibit the estrogen and androgen receptors. Estrogens and androgens have many important physiological functions not only for reproduction but also for the cardiovascular, immune, muscular, and nervous systems. Examples of chemical contaminants in water that affect sex hormone receptors are natural sex hormones, birth control pills, pharmaceuticals used to treat breast and prostate cancer, as well as isoflavones (so-called phytoestrogens) and certain chemicals used in plastic products.

In such an embodiment, the reporter cells could be cells that are stably transfected with a reporter plasmid, where the expression of a reporter protein is regulated by a promoter sequence containing elements that are responsive to compounds activating estrogen receptors or androgen receptors, respectively. Reporter cells transfected with such a reporter plasmid will respond to the presence of, for instance, estrogens or androgens in a water sample to be analyzed by increasing the production of the reporter protein. The presence of antiestrogens and antiandrogens, respectively, can be investigated by co-treatment of the reporter cells by a known inducer of the estrogen or androgen receptors, e.g., a known estrogen or androgen. Antagonistic effects towards the estrogen or androgen receptor will then be detected as a decrease in the reporter protein expression caused by the known inducer.

In these embodiments, the reference substance included in the first and second reference water samples could be an estrogen steroid hormone, such as estradiol, or an androgen steroid hormone, such as dihydrotestosterone.

Oxidative stress occurs from excess reactive oxygen radicals and an imbalance in the antioxidant defense system. It is a common mechanism behind various types of toxicity, such as teratogenicity and carcinogenicity. Many toxic substances, e.g., organic pollutants, certain pesticides, and natural substances can cause oxidative stress. Oxidative stress is also induced by disinfectant by-products (DBPs), which can be formed during water disinfection. Nearly 700 DBPs have been identified.

In such an embodiment, the reporter cells could be cells that are stably transfected with a reporter plasmid, where the expression of a reporter protein is regulated by a promoter sequence containing at least one antioxidant response element (ARE), such as at least one ARE that is responsive to nuclear factor erythroid 2-related factor 2 (NRF2). Reporter cells that are transfected with such a reporter plasmid will respond to the presence of oxidative stress causing compounds in a water sample to be analyzed by increasing the production of the reporter protein.

In these embodiments, the reference substance included in the first and second reference water samples could be a tert-butylhydroquinone.

When the aryl hydrocarbon receptor (AhR) is activated, metabolizing enzymes are induced and the effect of AhR activation is often called metabolic activation. The AhR has many physiological functions, including chemical and microbial defense, development, and in the regulation of inflammatory reactions. Many toxic substances activate the AhR, such as halogenated organic compounds, polycyclic aromatic hydrocarbons (PAHs), certain pesticides and pharmaceuticals, and naturally occurring substances, such as indoles and stilbenes.

In such an embodiment, the reporter cells could be cells that are stably transfected with a reporter plasmid, where the expression of a reporter protein is regulated by a promoter sequence containing elements that are responsive to substances activating AhR, such as dioxin response elements (DRE). Reporter cells that are transfected with such a reporter plasmid will respond to the presence of AhR activating compounds in a water sample to be analyzed by increasing the production of the reporter protein.

In these embodiments, the reference substance included in the first and second reference water samples could be a dioxin or a PAH.

Genotoxicity or DNA damage is a serious effect, which requires extensive testing and investigation for registration of chemicals, such as pesticides, food additives and flavorings. DNA damage in body cells can lead to cancer and to reproductive disorders when affecting germ cells.

In such an embodiment, the reporter cells could be cells that are stably transfected with a reporter plasmid, where the expression of a reporter protein is regulated by a promoter sequence containing elements that are responsive to, for instance, p53, i.e., a p53 responsive element. Reporter cells that are transfected with such a reporter plasmid will respond to the presence of genotoxic compounds in a water sample to be analyzed by increasing the production of the reporter protein.

In these embodiments, the reference substance included in the first and second reference water samples could be actinomycin D.

Thyroid hormone receptors are activated by binding of thyroid hormones. The receptors have important functions in the regulation of metabolism, heart rate, and organ development. Many environmental pollutants may interfere with the thyroid endocrine system and thereby cause adverse effects.

In such an embodiment, the reporter cells could be cells that are stably transfected with a reporter plasmid, where the expression of a reporter protein is regulated by a promoter sequence containing elements that are responsive to substances activating thyroid hormone receptors, such as thyroid response elements (TRE). Reporter cells that are transfected with such a reporter plasmid will respond to the presence of thyroid hormone receptor activating compounds in a water sample to be analyzed by increasing the production of the reporter protein.

In these embodiments, the reference substance included in the first and second reference water samples could be a thyroid hormone, for example triiodothyronine (T3).

Glucocorticoid receptors are activated by binding of glucocorticoids, such as cortisol. The receptor have important functions in the regulation of the immune system, development, and metabolism. Some environmental pollutants, such as pharmaceuticals, may interfere with the glucocorticoid endocrine system and thereby cause adverse effects.

In such an embodiment, the reporter cells could be cells that are stably transfected with a reporter plasmid, where the expression of a reporter protein is regulated by a promoter sequence containing elements that are responsive to substances activating the glucocorticoid receptor, such as glucocorticoid response elements (GRE). Reporter cells that are transfected with such a reporter plasmid will respond to the presence of glucocorticoid receptor activating compounds in a water sample to be analyzed by increasing the production of the reporter protein.

In these embodiments, the reference substance included in the first and second reference water samples could be a glucocorticoid, such as dexamethasone or cortisol.

The mineralocorticoid receptors are activated by binding of mineralocorticoids, such as aldosterone. The receptor have important functions in the regulation of cellular transport systems and thereby the reabsorption of sodium and maintenance of the normal salt balance. Some environmental pollutants, such as metals, pesticides, and pharmaceuticals, may interfere with the thyroid endocrine system and thereby cause adverse effects.

In such an embodiment, the reporter cells could be cells that are stably transfected with a reporter plasmid, where the expression of a reporter protein is regulated by a promoter sequence containing elements that are responsive to substances activating the mineralocorticoid receptor. Reporter cells that are transfected with such a reporter plasmid will respond to the presence of glucocorticoid receptor activating compounds in a water sample to be analyzed by increasing the production of the reporter protein.

In these embodiments, the reference substance included in the first and second reference water samples could be a mineralocorticoid, such as aldosterone.

Thus, in an embodiment, the reference substance is selected from the group consisting of a hormone, preferably a steroid hormone, and more preferably an estrogen steroid hormone, such as estradiol, an androgen steroid hormone, such as dihydrotestosterone, tert-butylhydroquinone, a dioxin, a polycyclic aromatic hydrocarbon, actinomycin D, a thyroid hormone, such as triiodothyronine (T3), a glucocorticoid, such as dexamethasone or cortisol, and a mineralocorticoid hormone, such as aldosterone.

In an embodiment, the processor 70 is configured to process the values generated by the detector 50 and generate, based on the value representative of the amount of the reporter protein expressed by the reporter cells in the first culture 11 of the multiple cultures 11, 13, 15, 16 of reporter cells, an output representative of a combined toxic effect of any hazardous compounds having a common mode of action in the water sample if the values representative of the amounts of the reporter protein expressed by the control cells in the first culture 12 and in the second culture 14 of the multiple cultures 12, 14 of control cells meet the first condition, and if the values representative of the amounts of the reporter protein expressed by the reporter cells in the second culture 13 and in the third culture 15 of the multiple cultures 11, 13, 15, 16 of reporter cells meet the second condition. In this embodiment, the common mode of action is selected from the group consisting of activation of sex hormone, thyroid hormone, glucocorticoid, and/or mineralocorticoid receptors, inhibition of sex hormone, thyroid hormone, glucocorticoid, and/or mineralocorticoid receptors, induction of oxidative stress, activation of aryl hydrocarbon receptor, genotoxicity, and any combination thereof.

The effect-based biosensor 1 of the invention is designed to be used on-site where water analysis is needed, such as at the site of a drinking water production plant, a wastewater treatment plant, or indeed any other facility at which water is processed or where the quality of water in terms of detecting presence of hazardous compounds is desired. The effect-based biosensor 1 may also be handled by users wanting to test the quality of their drinking water at home, such as testing water samples taken from a water well.

The effect-based biosensor 1 could be a portable, handheld device to be used by the staff at such plants or facilitates to test water samples. Alternatively, the effect-based biosensor 1 could be installed in connection with water tanks or basins at the plants or facilities to analyze water samples taken from the water tanks or basins. In such a case, the effect-based biosensor 1 could be used for continuous or at least intermittent monitoring of water quality at the plants or facilities.

The effect-based biosensor 1 could be used for water analysis of various water samples, such as analysis of drinking water quality, treatment efficiency in wastewater treatment plants, environmental monitoring of surface water or to evaluate new technical solutions in the water sector. The effect-based biosensor 1 may be also used on site for detection of chemical hazards at disasters, such as after floods or in polluted run-offs after fires.

In a typical use example, inputs to the effect-based biosensor 1 include a water sample to be analyzed, pure water as background control, a first reference sample comprising a maximum assay concentration of a reference substance, such as 17β-estradiol (E2), and a second reference sample comprising an effect-based trigger concentration of the reference substance. The effect-based trigger concentration corresponds to a “concentration of concern” for the reference substance, i.e., a maximum concentration of the reference substance that is regarded as being safe for the consumer or ecosystem and that would trigger preventive measures if it is exceeded.

In this use example, the reporter cells have been genetically modified for expression and release of NanoLuc® luciferase, which is 19.1 kDa, ATP-independent luciferase that utilizes a coelenterazine analog (furimazine) as substrate to produce high intensity, glow-type luminescence.

In this use example, the cell insert 10 comprises six different cell cultures as shown in FIG. 3. Four of these cell cultures 11, 13, 15, 16 are of the reporter cells genetically modified for expression and release of the NanoLuc® luciferase when exposed to compounds that activate a selected toxicity pathway, see Example 1. The amount of NanoLuc@ luciferase produced and released from the reporter cells is correlated to the amount of compounds that can activate the selected toxicity pathway. Two of the cell cultures 12, 14 preferably comprise the same type of cells but lack the regulatory element linking the expression of the NanoLuc@ luciferase to a specific toxicity pathway, such as lacking the EREs in Example 1. These control cells are instead constitutively expressing the NanoLuc® luciferase and are used to control cell viability/cytotoxicity.

The water sample to be tested is provided to two cell cultures 11, 12 as shown in FIG. 3, one culture 11 with reporter cells inducibly expressing NanoLuc® luciferase and one culture 12 with control cells lacking the regulatory element linking the expression of NanoLuc® luciferase to a specific toxicity pathway. The pure water control is also provided to a culture 13 of reporter cells and a culture 14 of control cells, whereas the two reference water samples are each provided to a respective culture 15, 16 of reporter cells.

The input water samples are mixed with concentrated cell culture medium added through a medium inlet 37. The concentrated cell culture medium comprises any components required by the reporter and control cells for cell culturing.

A portion of the supernatant, i.e., the cell culture medium, potentially comprising the produced and released NanoLuc® luciferase is, following an incubation period, transferred from respective cell culture 11-16 and forwarded to a respective detector chamber 51-56, to which a substrate, such as furimazine, is added through a substrate inlet 38. The luminescence is then measures by an optical detector 50.

The luminescence values as measured in the different detector chambers 51-56 are then used by the processor 70 for the interpretation of the analysis of the water sample. In more detail, the luminescence value from the detector chamber 55 with material from reporter cells exposed to the first reference water sample represents the maximum luminescence value (MAX). The luminescence value from the detector chamber 56 with material from reporter cells exposed to the second reference water sample represents the trigger concentration (T). The luminescence value from the detector chamber 53 with material from reporter cells exposed to pure water represents the minimum luminescence value (MIN). The luminescence value from the detector chamber 54 with material from the control cells exposed to pure water represents control cell viability (CCV). The corresponding luminescence value from the detector chamber 52 with material from the control cells exposed to the water sample represents assay cell viability (ACV). Finally, the luminescence value from the detector chamber 51 with material from the reporter cells exposed to the water sample represents the activity level to be determined.

The luminescence data as obtained from the six different detector chambers 51-56 is analyzed by the processor 70 according to the following algorithm in this particular use example.

The luminescence value of ACV must be at least a predefined percentage of the luminescence value of CCV. This initial check is used to verify that the reporter cells exposed to the water sample are sufficiently viable to get a meaningful interpretation of the luminescence values.

The luminescence value of MAX must be a predefined times larger than the luminescence value of MIN and the luminescence values of T must be below the luminescence value of MAX and above the luminescence value of MIN.

If the above-mentioned conditions are met, the luminescence value obtained for the water sample (represented by “•” in FIG. 3) is compared to the luminescence value of T. If the luminescence value obtained for the water sample is (equal to or) above the luminescence value of T, the combined toxic effects of hazardous compounds in the water sample is high, whereas if the luminescence value obtained for the water sample is (equal to or) below the luminescence value of T, the combined toxic effects of any hazardous compounds in the water sample is at acceptable levels.

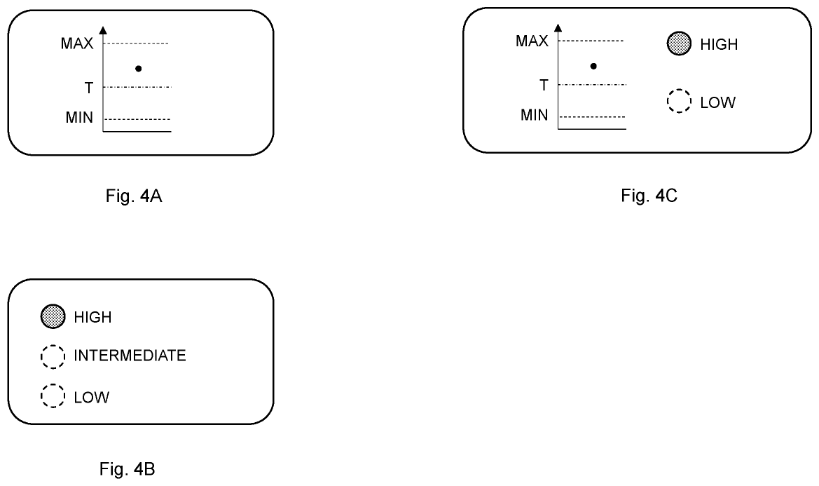

FIGS. 4A to 4C schematically illustrate presentation of output information generated by the processor 70. FIGS. 4A to 4C could represent the output information as presented on a display screen 78 of the effect-based biosensor 1, see FIG. 1A, or on a display 88 of an external device 80, see FIG. 2.

FIG. 4A illustrates an embodiment, in which the output shows the maximum control level (MAX), such as maximum luminescence value, as representing the amount of reporter protein produced by the third culture 15 of reporter cells, and the minimum control level (MIN), such as minimum luminescence value, as representing the amount of reporter protein produced by the second culture 13 of reporter cells. The output preferably also indicates the trigger level (T) as representing the amount of reporter protein produced by the fourth culture 16 of reporter cells. In this embodiment, the output also shows the value as obtained from the reporter cells exposed to the water sample to be analyzed in the first culture 11 of reporter cells. This value could, as in FIG. 4A, be represented by a dot •. The user could then determine whether this value • as above or below the trigger level T.

FIG. 4B illustrates another embodiment of presenting the output from the processor 70. In this embodiment, the output is representative of a comparison between the value V_{R1} representing the amount of reporter protein produced by the first culture 11 of reporter cells and the value V_{R4} representing the amount of reporter protein produced by the fourth culture 16 of reporter cells. In such a case, the output could be similar to traffic light with three different indications “high”, “intermediate” and “low”. For instance, the high indication is lit or activated when V_{R1}>V_{R4} and the low indication is lit or activated when V_{R1}<V_{R4}. The intermediate indication could then be lit or activated if V_{R1}≈V_{R4}, such as q×V_{R4}≤V_{R1}≤(2−q)×V_{R4} for some positive number q smaller than 1.

FIG. 4C illustrates a display of output form the processor 70 that is basically a combination of the graphical output in FIG. 4A and the traffic light output in FIG. 4B. However, in clear contrast to FIG. 4B, in this embodiment, the indications are either high, i.e., V_{R1}≤V_{R4}, or low, i.e., V_{R1}<V_{R4}. Alternatively, the indications as shown in FIG. 4B could be combined with the graphical output in FIG. 4A. It is also possible to present the output with merely the indications high and low, thereby omitting the intermediate indication in FIG. 4B.

### EXAMPLES

### Example 1

MCF-7/S0.5 human breast cancer cells (Sigma-Aldrich, catalogue no. SCC100) were cultured in medium supplemented with 1% charcoal-stripped fetal bovine serum (FBS) for 48 hours prior to seeding. The cells were then seeded in medium supplemented with 1% FBS and incubated for 48 hours. Then the culture medium was changed to one supplemented with 1% charcoal-stripped FBS and the cells were transfected with a reporter plasmid (pNL2.3 [secNluc/Hygro] from Promega, catalogue no. N1081) encoding NanoLuc® luciferase with an N-terminal secretion signal under transcriptional control of an estrogen responsive promoter comprising four upstream repeats of an estrogen response element (ERE) (SEQ ID NO: 1, 5′-CGAGAGCTAAAATAACACATTCAG-3′, Molecular and Cellular Endocrinology (2000) 165 (1-2): 151-161).

The luminescence was measured in the cell culture medium by a Spark® Multimode Microplate Reader (Tecan) from three experimental groups: a) negative control with only cell culture medium but no cells, b) cell culture medium and non-transfected cells, and c) cell cultured medium and cells transfected with the reporter plasmid mentioned above (“reporter cells”).

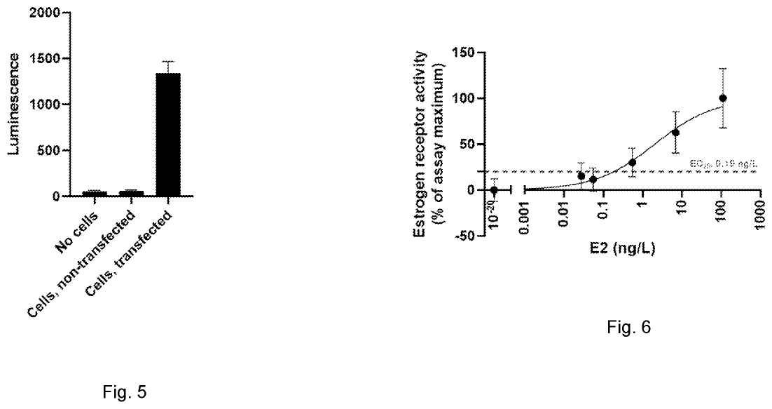

The transfection was successful, the reporter cells produced luciferase in a detectable amount and the luciferase was secreted into the cell culture medium, i.e., no need for cell lysis, see FIG. 5. The background luminescence (no cell control and non-transfected control) was less than 5% of the luminescence of the reporter cells.

### Example 2

The MCF-7/S0.5 cells transfected with the reporter plasmid where the expression of NanoLuc® luciferase with an N-terminal secretion signal was under the regulation of an estrogen responsive promoter produced in Example 1 were exposed for 24 h to increasing concentrations of 17β-estradiol, ranging from 0.03 to 109 ng/L. The luminescence was then analyzed in the cell culture medium, without cell lysis.

The amount of NanoLuc® luciferase secreted into the cell culture medium was increasing with increasing concentration of E2, see FIG. 6. It was possible to measure the luminescence without cell lysis. The effect concentration 20% (EC20) was 0.19 ng/L E2, which was in the desirable range to reach the sensitivity needed for applications both in wastewater treatment plants and drinking water treatment plants.

### Example 3

The MCF-7/S0.5 cells transfected with the reporter plasmid where the expression of NanoLuc® luciferase with an N-terminal secretion signal was under the regulation of an estrogen responsive promoter produced in Example 1 were exposed for 24 h to either 0 ng/L (assay minimum), 0.54 ng/L (predefined trigger level) or 109 ng/L (assay maximum) of E2. In addition, eight water samples were analyzed: four water samples with low levels of estrogens (a concentration below 0.54 ng/L of E2) and four water samples with high levels of estrogens (a concentration above 0.54 ng/L of E2). The luminescence was then analyzed in the cell culture medium, without cell lysis.

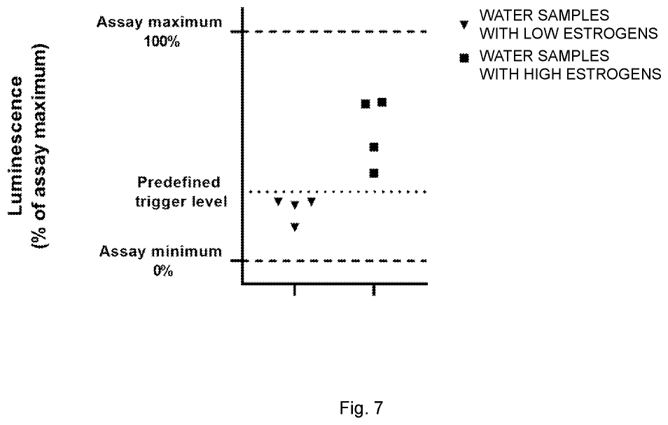

Assay minimum, assay maximum and predefined trigger level could be established, see FIG. 7. All eight water samples had a NanoLuc® luciferase activity that was higher than assay minimum but lower than assay maximum. All four water samples with high levels of estrogens had a NanoLuc@ luciferase activity that was higher than the activity of the predefined trigger level. All four water samples with low levels of estrogens had a NanoLuc® luciferase activity that was lower than the activity of the predefined trigger level. Hence, by comparing the NanoLuc® luciferase activity of a water sample, with the luciferase activity of the predefined trigger level, it was possible to determine if that water sample contained estrogenic activity corresponding to a bioequivalent concentration that was above or below the predefined trigger level.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1-27. (canceled)

28. An effect-based biosensor for water analysis comprising:
a cell insert comprising multiple cultures of reporter cells genetically modified to respond to presence of hazardous compounds by expressing a reporter protein and multiple cultures of control cells genetically modified to constitutively express the reporter protein;
an inlet system in fluid communication with the cell insert and comprising:
at least one first inlet configured to receive a water sample to be analyzed and in fluid communication with a first culture of the multiple cultures of reporter cells and a first culture of the multiple cultures of control cells;
at least one second inlet configured to receive a control water sample and in fluid communication with a second culture of the multiple cultures of reporter cells and a second culture of the multiple cultures of control cells;
a third inlet configured to receive a first reference water sample comprising a first concentration of a reference substance and in fluid communication with a third culture of the multiple cultures of reporter cells;
a detector configured to generate, for each culture of the multiple cultures of reporter cells, a value representative of an amount of the reporter protein expressed by the reporter cells and, for each culture of the multiple cultures of control cells, a value representative of an amount of the reporter protein expressed by the control cells; and
a processor connected to the detector and configured to process the values generated by the detector and generate, based on the value representative of the amount of the reporter protein expressed by the reporter cells in the first culture of the multiple cultures of reporter cells, an output representative of a combined toxic effect of any hazardous compounds in the water sample if the values representative of the amounts of the reporter protein expressed by the control cells in the first culture and in the second culture of the multiple cultures of control cells meet a first condition, and if the values representative of the amounts of the reporter protein expressed by the reporter cells in the second culture and in the third culture of the multiple cultures of reporter cells meet a second condition.

29. The biosensor according to claim 28, wherein the first condition is a cell viability condition, and
wherein the second condition is a responsiveness condition.

30. The biosensor according to claim 29, wherein the first condition is a cell viability condition indicating that the water sample is not cytotoxic for the reporter cells and the control cells, and
wherein the second condition is a responsiveness condition indicating that the effect-based biosensor can produce range of values representative of different amounts of reporter protein.

31. The biosensor according to claim 28, wherein the processor is configured to:
compare the values representative of the amounts of the reporter protein expressed by the control cells in the first culture and in the second culture of the multiple cultures of control cells; and
determine that the first condition is met if the value representative of the amount of the reporter protein expressed by the control cells in the first culture of the multiple cultures of control cells is at least a predefined percentage of the value representative of the amount of the reporter protein expressed by the control cells in the second culture of the multiple cultures of control cells.

32. The biosensor according to claim 28, wherein the processor is configured to:
compare the values representative of the amounts of the reporter protein expressed by the reporter cells in the second culture and in the third culture of the multiple cultures of reporter cells; and
determine that the second condition is met if the value representative of the amount of the reporter protein expressed by the reporter cells in the third culture of the multiple cultures of reporter cells is at least a predefined times larger than the value representative of the amount of the reporter protein expressed by the reporter cells in the second culture of the multiple cultures of reporter cells.

33. The biosensor according to claim 28, wherein the processor is configured to generate, based on the value representative of the amount of the reporter protein expressed by the reporter cells in the first culture of the multiple cultures of reporter cells, the output if the values representative of the amounts of the reporter protein expressed by the control cells in the first culture and in the second culture of the multiple cultures of control cells meet the first condition, if the values representative of the amounts of the reporter protein expressed by the reporter cells in the second culture and in the third culture of the multiple cultures of reporter cells meet the second condition, and if the values representative of the amount of the reporter protein expressed by the reporter cells in the first culture and the second culture of the multiple cultures of reporter cells meet a third condition.

34. The biosensor according to claim 33, wherein the processor is configured to:
compare the value representative of the amount of the reporter protein expressed by the reporter cells in the first culture of the multiple cultures of reporter cells with the value representative of the amount of the reporter protein expressed by the reporter cells in the second culture of the multiple cultures of reporter cells; and
determine that the third condition is met if the value representative of the amount of the reporter protein expressed by the reporter cells in the first culture of the multiple cultures of reporter cells is equal to or above the value representative of the amount of the reporter protein expressed by the reporter cells in the second culture of the multiple cultures of reporter cells.

35. The biosensor according to claim 34, wherein the processor is configured to:
compare the value representative of the amount of the reporter protein expressed by the reporter cells in the first culture of the multiple cultures of reporter cells with the values representative of the amounts of the reporter protein expressed by the reporter cells in the second culture and in the third culture of the multiple cultures of reporter cells; and
determine that the third condition is met if the value representative of the amount of the reporter protein expressed by the reporter cells in the first culture of the multiple cultures of reporter cells is below the value representative of the amount of the reporter protein expressed by the reporter cells in the third culture of the multiple cultures of reporter cells but equal to or above the value representative of the amount of the reporter protein expressed by the reporter cells in the second culture of the multiple cultures of reporter cells.

36. The biosensor according to claim 28, wherein the inlet system comprises a fourth inlet configured to receive a second reference water sample comprising a second concentration of the reference substance that is lower than the first concentration and in fluid communication with a fourth culture of the multiple cultures of reporter cells, and
wherein the processor is configured to:
compare the value representative of the amount of the reporter protein expressed by the reporter cells in the first culture of the multiple cultures of reporter cells and the value representative of the amount of the reporter protein expressed by the reporter cells in the fourth culture of the multiple cultures of reporter cells; and
generate the output based on the comparison if the values representative of the amounts of the reporter protein expressed by the control cells in the first culture and in the second culture of the multiple cultures of control cells meet the first condition, and if the values representative of the amounts of the reporter protein expressed by the reporter cells in the second culture and in the third culture of the multiple cultures of reporter cells meet the second condition.

37. The biosensor according to claim 36, wherein the processor is configured to generate an output indicating that the combined toxic effect of any hazardous compounds in the water sample is unacceptably high if the value representative of the amount of the reporter protein expressed by the reporter cells in the first culture of the multiple cultures of reporter cells is higher than, or equal to or higher than, the value representative of the amount of the reporter protein expressed by the reporter cells in the fourth culture of the multiple cultures of reporter cells.

38. The biosensor according to claim 36, wherein the processor is configured to generate an output indicating that the combined toxic effect of any hazardous compounds in the water sample is acceptable if the value representative of the amount of the reporter protein expressed by the reporter cells in the first culture of the multiple cultures of reporter cells is equal to or lower than, or lower than, the value representative of the amount of the reporter protein expressed by the reporter cells in the fourth culture of the multiple cultures of reporter cells.

39. The biosensor according to claim 36, wherein the processor is configured to generate the output if the values representative of the amounts of the reporter protein expressed by the control cells in the first culture and in the second culture of the multiple cultures of control cells meet the first condition, if the values representative of the amounts of the reporter protein expressed by the reporter cells in the second culture and in the third culture of the multiple cultures of reporter cells meet the second condition, and if the values representative of the amounts of the reporter protein expressed by the reporter cells in the second culture, the third culture, and the fourth culture of the multiple cultures of reporter cells meet a fourth condition.

40. The biosensor according to claim 39, wherein the processor is configured to:
compare the value representative of the amount of the reporter protein expressed by the reporter cells in the fourth culture of the multiple cultures of reporter cells with the values representative of the amounts of the reporter protein expressed by the reporter cells in the second culture and in the third culture of the multiple cultures of reporter cells; and
determine that the fourth condition is met if the value representative of the amount of the reporter protein expressed by the reporter cells in the fourth culture of the multiple cultures of reporter cells is below the value representative of the amount of the reporter protein expressed by the reporter cells in the third culture of the multiple cultures of reporter cells but above the value representative of the amount of the reporter protein expressed by the reporter cells in the second culture of the multiple cultures of reporter cells.

41. The biosensor according to claim 28, wherein the cell insert comprises multiple cultures of reporter cells genetically modified to respond to presence of hazardous compounds by expressing and extracellularly releasing the reporter protein and multiple cultures of control cells genetically modified to constitutively express and extracellularly release the reporter protein.

42. The biosensor according to claim 28, wherein the inlet system further comprises a substrate inlet configured to receive a substrate that is converted by the reporter protein into a product upon emission of light, and
wherein the detector is an optical detector configured to detect emitted light.

43. The biosensor according to claim 42, wherein the reporter protein is a luciferase.

44. The biosensor according to claim 28, wherein the reporter cells and the control cells are selected from the group consisting of yeast cells, fish cells and mammalian cells.

45. The biosensor according to claim 44, wherein the reporter cells and the control cells are human cells.

46. The biosensor according to claim 28, wherein the reporter cells are of a same type of cells as the control cells.

47. The biosensor according to claim 28, wherein the reporter cells comprise a reporter gene encoding the reporter protein, wherein the reporter gene is under transcriptional control of an inducible promoter configured to induce expression of the reporter gene when the reporter cells are exposed to hazardous compounds, and
wherein the control cells comprise the reporter gene under transcriptional control of a constitutive promoter.

48. The biosensor according to claim 28, wherein the inlet system comprises at least one medium inlet configured to receive a culture medium for the report cells and the control cells, and
wherein the at least one medium inlet is in fluid communication with the multiple cultures of reporter cells and the multiple cultures of control cells.

49. The biosensor according to claim 28, further comprising a display screen configured to display the output or a parameter derived based on the output.

50. The biosensor according to claim 28, further comprising a transmitter configured to wirelessly transmit the output or a parameter derived based on the output to an external device.

51. The biosensor according to claim 28, wherein the reporter cells are genetically modified to respond to presence of hazardous compounds having a common mode of action resulting in activation of an adverse outcome pathway in the reporter cells.

52. The biosensor according to claim 51, wherein the reporter cells comprise a reporter gene encoding the reporter protein, wherein the reporter gene is under transcriptional control of an inducible promoter, and
wherein activation of the inducible promoter is a molecular event of the adverse outcome pathway.

53. The biosensor according to claim 51, wherein the reference substance is capable of activating the adverse outcome pathway in the reporter cells.

54. The biosensor according to claim 28, wherein the reference substance is selected from the group consisting of a hormone, a dioxin, a polycyclic aromatic hydrocarbon, actinomycin D, tert-butylhydroquinone, triiodothyronine, dexamethasone, cortisol, and aldosterone.

55. The biosensor according to claim 54, wherein the reference substance is selected from the group consisting of an estrogen steroid hormone and an androgen steroid hormone.

56. The biosensor according to claim 28, wherein the processor is configured to process the values generated by the detector and generate, based on the value representative of the amount of the reporter protein expressed by the reporter cells in the first culture of the multiple cultures of reporter cells, an output representative of a combined toxic effect of any hazardous compounds having a common mode of action in the water sample if the values representative of the amounts of the reporter protein expressed by the control cells in the first culture and in the second culture of the multiple cultures of control cells meet the first condition, and if the values representative of the amounts of the reporter protein expressed by the reporter cells in the second culture and in the third culture of the multiple cultures of reporter cells meet the second condition, wherein the common mode of action is selected from the group consisting of activation of sex hormone, thyroid hormone, glucocorticoid, and/or mineralocorticoid receptors, inhibition of sex hormone, thyroid hormone, glucocorticoid, and/or mineralocorticoid receptors, induction of oxidative stress, activation of aryl hydrocarbon receptor, genotoxicity, and any combination thereof.

57. The biosensor according to claim 28, wherein the cell insert comprises multiple cultures of reporter cells genetically modified to respond to presence of hazardous compounds having a common mode of action on the reporter cells by expressing the reporter protein, wherein the common mode of action is selected from the group consisting of activation of sex hormone, thyroid hormone, glucocorticoid, and/or mineralocorticoid receptors, inhibition of sex hormone, thyroid hormone, glucocorticoid, and/or mineralocorticoid receptors, induction of oxidative stress, activation of aryl hydrocarbon receptor, genotoxicity, and any combination thereof.
